(19) United States
(12) Patent Application Publication
(10) Pub. No.: US 2020/0103322 A1
(43) Pub. Date: Apr. 2, 2020

(54) Test Fixtures for Evaluating Mechanical Properties of Asphalt Samples and Related Systems and Methods

(71) Applicant: InstroTek, Inc., Raleigh, NC (US)

(72) Inventors: Ali Regimand, Raleigh, NC (US); Ethan Steckmann, Durham, NC (US); Andrew LaCroix, Raleigh, NC (US); Richard Stacy, Fayetteville, NC (US); Lawrence James, Raleigh, NC (US); Dirk Steckmann, Raleigh, NC (US)

(21) Appl. No.: 16/148,540

(22) Filed: Oct. 1, 2018

(51) Int. Cl. G01N 3/08; G01N 3/06

(52) U.S. Cl. CPC G01N 3/08; G01N 33/42; G01N 3/066

(57) ABSTRACT

A system for evaluating properties of an asphalt sample includes a load frame and a test fixture. The load frame includes a platform and a loading rod. The test fixture includes: a base configured to rest on the platform of the load frame; first and second spaced apart vertical guide bars extending upwardly from the base; a horizontal cross bar above the base and extending between the first and second guide bars, wherein the asphalt sample is configured to be held between the base and the cross bar; a load plate above the cross bar, the load plate configured to receive the loading rod of the load frame to apply a load to the asphalt sample; a load cell above the base and configured to measure the applied load and to generate corresponding load electrical signals; and a controller configured to receive the load electrical signals.

### BACKGROUND

Determination of compacted material strength is a routine test that is performed in the construction industry. In the asphalt material design and quality control, indirect tensile strength, crack susceptibility and tension tests are conducted to ascertain material properties and performance of pavements. These tests have been in existence for decades in research and production of asphalt materials. To conduct these tests, a load frame capable of applying load is used along with a fixture that is used to hold the sample and to transfer the load, either in a compression tension, or indirect tension configuration. The load frame is typically programmed to travel at a predetermined speed, e.g., 50 mm per minute, and uses an integrated load cell to output the load. The output is either stored digitally or plotted on paper. Some load frames also provide displacement instruments (e.g., linear variable differential transducer (LVDT), potentiometer, non-contact magnetic displacement devices, etc.) to measure and output displacement measurements. For each of the varying tests conducted on the load frame, a specific fixture is provided. The fixtures are primarily provided to hold the samples in a stable position and to consistently direct the load, compression or tension, onto the sample to ensure accuracy and repeatability of the data.

Conventional load frames do not have the capability to collect data at a high rate to allow for calculation of different performance measures required in the industry. Furthermore, some older frames are analog machines and only provide plotting and printing ability, which makes them unusable for many of the performance tests now required in the industry, even though they may be capable of loading the samples at the required rate. For this reason, many users buy specific load frames to conduct each specific test.

### SUMMARY

Some embodiments of the present invention are directed to a system for evaluating properties of an asphalt sample. The system includes a load frame including a platform and a loading rod. One of the platform and the loading rod is configured to translate up and down away from and toward the other one of the platform and the loading rod. The system includes a test fixture including: a base configured to rest on the platform of the load frame; first and second spaced apart vertical guide bars extending upwardly from the base; a horizontal cross bar above the base and extending between the first and second guide bars, wherein the asphalt sample is configured to be held between the base and the cross bar; a load plate above the cross bar, the load plate configured to receive the loading rod of the load frame to apply a load to the asphalt sample; a load cell above the base and configured to measure the applied load and to generate corresponding load electrical signals; and a controller configured to receive the load electrical signals.

In some embodiments, the cross bar is an upper cross bar, and the test fixture further includes a horizontal lower cross bar above the base and extending between the first and second guide bars. The load plate may be on the upper cross bar and the load cell may be between the base and the lower cross bar. The test fixture may further include a lower press bar at an upper portion or surface of the lower cross bar and an upper press bar at a lower portion or surface of the upper cross bar, and the asphalt sample may be configured to be received between the lower press bar and the upper press bar.

In some embodiments, the asphalt sample is cylindrical and the lower press bar and the upper press bar are each arcuate to surround at least a major portion of a circumference of the asphalt sample.

In some embodiments, the test fixture further includes first and second upper guide bearings each coupled to the upper cross bar. The first upper guide bearing may surround the first guide bar and the second upper guide may surround the second guide bar. The first and second upper guide bearings may be configured to allow vertical movement of the upper guide bar upon application of the load.

In some embodiments, the test fixture further includes a transmitter or transceiver. The controller may be configured to, using the transmitter or transceiver, wirelessly transmit load data associated with the load electrical signals to an electronic device. In some embodiments, the system further includes the electronic device. The electronic device may be configured to display the load data versus displacement data and optionally a peak load to break the asphalt sample.

In some embodiments, the test fixture further includes a horizontal load bar above the cross bar and extending between the first and second guide bars. The load plate may be on the load bar and/or the load cell may be between the cross bar and the load bar. The asphalt sample may be semi-cylindrical with a circumference including a curved portion and a flat portion. The test fixture may further include: a press bar at a lower portion or surface of the cross bar and configured to engage the curved portion of the circumference of the asphalt sample; and/or first and/or second rollable pins above the base and configured to engage the flat portion of the circumference of the asphalt sample thereon.

In some embodiments, the test fixture further includes a first displacement transducer coupled to a first side of the cross bar adjacent the first guide bar and/or a second displacement transducer coupled to a second side of the cross bar adjacent the second guide bar. The first and/or second displacement transducer may be configured to measure a displacement of the cross bar as the load is applied by the load frame and to generate corresponding displacement electrical signals. The load cell may be configured to generate the load electrical signals and/or the first and second displacement transducers may be configured to generate the displacement electrical signals at a rate of 40 Hz or greater.

In some embodiments, the first displacement transducer includes a first plunger and the second displacement transducer comprises a second plunger. A first shelf may be coupled to the first guide bar and a second shelf may be coupled to the second guide bar. The first plunger may rest on the first shelf and the second plunger may rest on the second shelf.

In some embodiments, the test fixture further includes a transmitter or transceiver. The controller may be configured to receive the displacement electrical signals from the first and second displacement transducers. The controller may be configured to, using the transmitter or transceiver, wirelessly transmit displacement data associated with the displacement electrical signals and load data associated with the load electrical signals to an electronic device.

In some embodiments, the system further includes the electronic device including a controller and/or a display. The controller of the test fixture or the controller of the electronic device may be configured to determine a fracture energy of the asphalt sample based on the load data and the displacement data and/or to determine a brittleness of the asphalt sample based on the load data and the displacement data. The controller of the electronic device may be configured to direct the display to display the load data, the displacement data, the fracture energy of the asphalt sample, and/or the brittleness of the asphalt sample.

Some other embodiments of the present invention are directed to a test fixture for use with a load frame and for evaluating properties of an asphalt sample. The test fixture includes: a base configured to rest on a platform of the load frame; first and second spaced apart vertical guide bars extending upwardly from the base; a horizontal cross bar above the base and extending between the first and second guide bars, wherein the asphalt sample is configured to be held between the base and the cross bar; a load plate above the cross bar, the load plate configured to receive a loading rod of the load frame to apply a load to the asphalt sample; a load cell above the base and configured to measure the load and to generate corresponding load electrical signals; and a controller configured to receive the load electrical signals.

In some embodiments, the cross bar is an upper cross bar and the load plate is on the upper cross bar. The test fixture may include a horizontal lower cross bar above the base and extending between the first and second guide bars, wherein the load cell is between the base and the lower cross bar. The test fixture may include a lower press bar at an upper portion or surface of the lower cross bar and an upper press bar at a lower portion or surface of the upper cross bar, wherein the asphalt sample is configured to be received between the lower press bar and the upper press bar. The test fixture may include a transmitter or transceiver. The controller may be configured to, using the transmitter or transceiver, wirelessly transmit load data associated with the load electrical signals to an electronic device such that the electronic device can store and/or display the load data versus displacement data and optionally a peak load to break the asphalt sample.

In some embodiments, the test fixture further includes: a horizontal load bar above the cross bar and extending between the first and second guide bars, wherein the load plate is on the load bar and the load cell is between the cross bar and the load bar; at least one displacement transducer coupled to the cross bar and configured to measure a displacement of the cross bar as the load is applied by the load frame and to generate corresponding displacement electrical signals; and/or a transmitter or transceiver. The controller may be configured to receive the displacement electrical signals from the first and second displacement transducers. The controller may be configured to, using the transmitter or transceiver, wirelessly transmit displacement data associated with the displacement electrical signals and load data associated with the load electrical signals to an electronic device such that the electronic device can store and/or display the load data, the displacement data, a fracture energy of the asphalt sample based on the load data and the displacement data, and/or a brittleness of the asphalt sample based on the load data and the displacement data. The controller may be configured to, using the transmitter or transceiver, wirelessly transmit the displacement data and load data to the electronic device at a rate of at least 40 Hz.

Some other embodiments of the present invention are directed to a method of evaluating mechanical properties of an asphalt test sample. The method includes: providing a test fixture comprising a base, first and second spaced apart vertical guide bars extending upwardly from the base, a horizontal cross bar above the base and extending between the first and second guide bars, a load plate above the cross bar, a load cell above the base, a controller in communication with the load cell, and/or a transmitter in communication with the controller; positioning an asphalt sample between the base and the cross bar of the test fixture; positioning the test fixture in a load frame by resting the base of the test fixture on a platform of the load frame; loading the asphalt sample by receiving a loading rod of the test frame on the load plate of the test fixture; generating load electrical signals using the load cell in response to the loading step; receiving the load electrical signals at the controller; and transmitting, optionally wirelessly, load data associated with the load electrical signals to an electronic device using the controller and optionally the transmitter.

In some embodiments, the method further includes displaying and/or storing at the electronic device the load data versus displacement data and optionally a peak load to break the asphalt sample.

In some embodiments, the test fixture further includes the ability to measure the relative displacement of the test fixture to the load frame. A preferred embodiment is the displacement transducer(s) are coupled to the body of the jig and are referenced to rod(s) which are coupled to the cross bar of the loading frame. The method may further include: generating displacement electrical signals using the first and/or second displacement transducers in response to the loading step; receiving the displacement electrical signals at the controller; transmitting, optionally wirelessly, displacement data associated with the displacement electrical signals to the electronic device using the controller and optionally the transmitter; and/or displaying and/or storing at the electronic device the load data, the displacement data, a fracture energy of the asphalt sample based on the load data and the displacement data, and/or a brittleness of the asphalt sample based on the load data and the displacement data. The steps of transmitting the load data and transmitting the displacement data may be carried out at a rate of 40 Hz or more.

Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE FIGURES

FIG. 1A is a front perspective view of a load frame according to some embodiments of the invention.

FIG. 1B is a schematic diagram of the load frame of FIG. 1A with a test fixture or jig according to embodiments described herein.

FIG. 2A is a front view of a test fixture or jig according to some embodiments of the invention. The jig may be a Smart TSR jig configured for use in a tensile strength ratio (TSR) test.

FIG. 2B is a side view of the test fixture or jig of FIG. 2A.

FIG. 3 is a schematic diagram of the test fixture or jig of FIG. 2A configured to wirelessly communicate with an electronic device according to some embodiments of the invention.

FIG. 4A is a front view of another test fixture or jig according to some embodiments of the invention. The jig may be a Smart SCB jig configured for use in a semi-circular bend (SCB) test.

FIG. 4B is a side view of the test fixture or jig of FIG. 4A.

FIG. 5 is a schematic diagram of the test fixture or jig of FIG. 4A configured to wirelessly communicate with an electronic device according to some embodiments of the invention.

FIG. 6A is a front view of yet another test fixture or jig according to some embodiments of the invention. The jig may be a Smart Marshall Stability jig configured for use in a Marshall Stability test.

FIG. 6B is a side view of the test fixture or jig of FIG. 6A.

FIG. 7 is an illustration of data that can be collected and output using the test fixtures or jigs of FIGS. 2A, 4A, and 6A according to some embodiments of the invention.

FIG. 8 is a front view of yet another test fixture or jig according to some embodiments of the invention. The jig may be a Smart TSR jig configured for use in a tensile strength ratio (TSR) test that requires measuring the displacement as well as the load, such as the IDEAL cracking test.

### DETAILED DESCRIPTION

The present invention now will be described more fully hereinafter with reference to the accompanying drawings, in which illustrative embodiments of the invention are shown. In the drawings, the relative sizes of regions or features may be exaggerated for clarity. This invention may, however, be embodied in many different forms and should not be construed as limited to the embodiments set forth herein; rather, these embodiments are provided so that this disclosure will be thorough and complete, and will fully convey the scope of the invention to those skilled in the art.

It will be understood that when an element is referred to as being “coupled” or “connected” to another element, it can be directly coupled or connected to the other element or intervening elements may also be present. In contrast, when an element is referred to as being “directly coupled” or “directly connected” to another element, there are no intervening elements present. Like numbers refer to like elements throughout. As used herein the term “and/or” includes any and all combinations of one or more of the associated listed items.

In addition, spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly.

Well-known functions or constructions may not be described in detail for brevity and/or clarity.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises,” “includes,” “comprising,” and/or “including,” when used in this specification, specify the presence of stated features, integers, steps, operations, elements, and/or components, but do not preclude the presence or addition of one or more other features, integers, steps, operations, elements, components, and/or groups thereof.

It is noted that any one or more aspects or features described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The term “automatically” means that the operation is substantially, and may be entirely, carried out without human or manual control, direction and/or input, and can be programmatically directed or carried out.

The term “programmatically” refers to operations directed and/or primarily carried out electronically by computer program modules, code and/or instructions.

According to some embodiments, the present invention provides a fixture that holds the samples in a fixed position, which ensures the load is directed consistently onto the sample. The fixture is independently instrumented with a load measuring instrument, i.e., load cell, and/or displacement measurement instruments (one or more), depending on the required test. The fixture is designed to work with most load frames and uses independent electronics to measure, collect and transfer data to a handheld readout device or a computer. The data can be transferred wirelessly (e.g., via Bluetooth or RF) or by a wire connection. The data can normally be collected at frequencies required in test standards, such as 20, 40, 50, or 100 Hz or arranged so that collection may occur at any frequency up to 200 Hz and higher and all necessary calculations can be programmed and displayed on a computer, or a portable electronic device using, e.g., Android or IOS platform. The present invention allows researchers, engineers and technical professionals the ability to use their existing load frames to conduct all the performance tests that require load and displacement data, which can result in savings of thousands of dollars. Common load frames are fitted with a load cell so that the force can be recorded. In addition, the load frame may be fitted with a mechanism to monitor the vertical displacement as the load is being applied. In many of the older, but still commonly used load frames, this data is recorded by using an x-y pen plotter. The plotter paper is moved along the x-axis at a constant rate as an indication of distance. At the same time, the y-axis is moved by the amplitude of the electrical signal of the load cell which is converted to a height on the plotter. Furthermore, those skilled in the art can appreciate that new performance tests developed in the industry can be implemented at the user site by incorporating the electronic and the measurement instruments of the present invention onto a new fixture, allowing the use of the fixture(s) with existing load frames in the market.

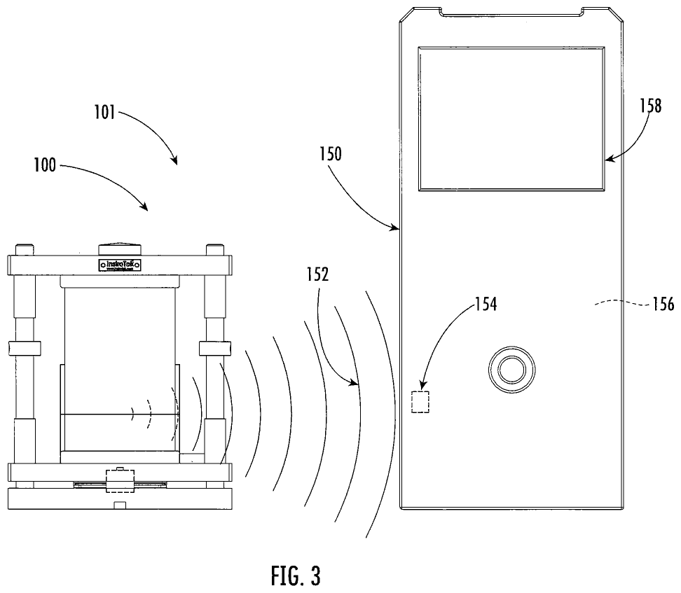

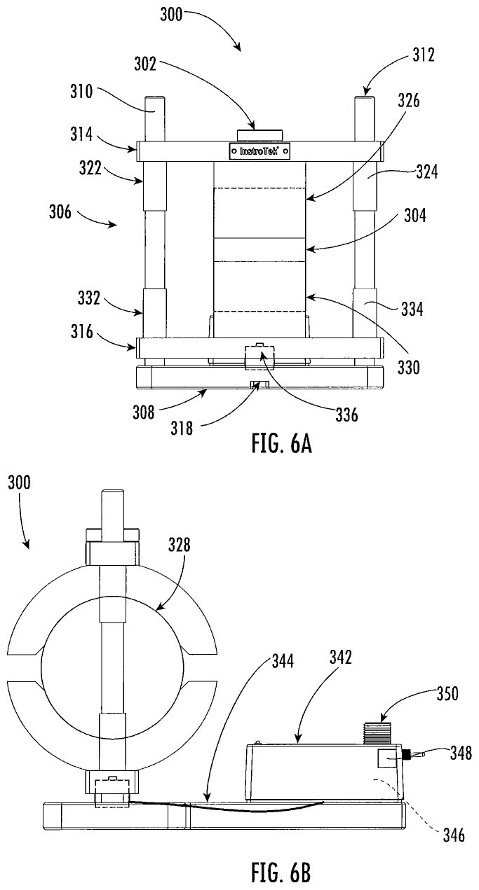

Though not exhaustive, embodiments of the present invention can be used to acquire and analyze the data of the following tests currently used in performance evaluations of asphalt mixture:
1—Moisture Sensitivity—This test includes conditioning a set of compacted samples for a predetermined time in hot water followed by freezing the samples (conditioned samples) and providing another set at room temperature (unconditioned samples). The samples tested are cylindrical compacted samples approximately 100 or 150 mm in diameter and 63.5 or 100 mm in thickness. Each sample is placed in a test fixture, commonly referred to as the Lottman or TSR breaking head, which includes a base, two posts, a lower blade, an upper cross bar that is guided by the posts, and a second blade on the underside of the cross bar. Both blades have a thickness of about a half inch and are machined to fit the circumference of the core or sample. The asphalt core is placed in the test fixture so that the lower blade is in contact with one cylindrical side of the sample and the upper blade is in contact with the opposite cylindrical side of the sample. The uninstrumented test fixture and sample are then placed into a load or loading frame that will apply compressive load on the sample between the lower and upper blades. The compressive load along the diameter of the sample causes an outward tensile force due to the expansion of the material perpendicular to the compressive force. This behavior is called the indirect tensile mode of loading. The Indirect Tensile Strength for both the conditioned and unconditioned samples is determined by placing each sample in a fixture and applying a load by moving a loading head of the load frame at 1.5 to 50 mm per minute. The peak load to break each sample is determined from a plot, printout or downloaded data produced by the load frame. The ultimate result of the test is the tensile strength ratio (TSR), which is the ratio of the conditioned strength to the unconditioned strength.
2—Marshall Stability Test—This test has been commonly used during asphalt mixture design. The standard for this test method is ASTM D6927 or AASHTO T 245. The test involves using a test fixture with two halves of a steel pipe that surround the sample. A compressive load is applied to the top of the test fixture, which creates an indirect tensile force on the specimen, like the Lottman breaking head, but the larger contact area provides resistance or confinement to the mixture so the mixture is compressed instead of split in half. The testing speed is 50 mm per minute. The test requires measuring the peak load (stability) and the displacement at the peak load (flow number, which has units of 0.01 inches).
3—Fracture Energy and Slope of Indirect Tension Specimens—This type of test uses a similar sample and setup as the test for moisture sensitivity but the test is used to estimate the cracking potential of asphalt mixtures. Estimation of the peak load may have been acceptable for determining the maximum strength of the asphalt mixtures for moisture sensitivity, but these types of tests require more detailed information. Several performance measures, such as the facture energy and the slope after the peak load, are used to characterize the fracture behavior of asphalt mixtures. For example, the indirect tension asphalt cracking test (IDEAL-CT) requires calculating the fracture energy, i.e., the area under the load versus displacement curve, for the whole loading history. Furthermore, the slope after the peak load is used to determine the brittleness of the mixture. A steeper slope represents a more brittle mixture. Both the fracture energy and slope measurements require data gathered at rates greater than 20 Hz to accurately determine the parameters needed.
4—Semi-circular bend (SCB) test—This test, which originally was developed for testing metal samples, has become a popular test to predict the cracking potential of asphalt mixtures over the last decade. Several versions of the test currently exist, such as ASTM D8044 and AASHTO TP124, but both methods measure the fracture energy and the brittleness of semi-circular samples. These properties are determined from the load versus displacement curves (FIG. 7) of semi-circular samples with a notch in the center of the flat face. The facture energy is measured by placing each sample in a fixture designed for 3 point bending (FIG. 4A) and applying a compressive load by moving a load head of the load frame at a speed between 0.5 and 50 mm per minute.

These above tests provide a sample of test fixtures for which embodiments of the present invention can be used. However, those skilled in the art will understand other uses to instrument other test fixtures/jigs to collect and analyze data for specific tests.

Measuring and calculating the results of the above-described tests can be improved using embodiments of the present invention. The present invention provides the following solutions for challenges of performing these tests in conventional load frames.
1) Plotter parts, including plotter pins and replacement parts, are becoming very expensive and hard to find, which makes properly operating load frames useless. The present invention provides an alternative that electronically stores the data instead of physically plotting. Plotting can be done through a software application and printed on any printer or provided digitally to a display and/or database record.
2) Electronic storage of data solves another major drawback of plotting, which is determining and transcribing the results for further analysis. If the user wishes to perform further analysis from a plot, the data has to be extracted from the hard copy. This can be a difficult, time consuming process with inherent inaccuracy, since the user is essentially estimating values from a graph. For example, the peak compressive load is required to determine the indirect tensile strength of moisture conditioned samples. The fixtures according to embodiments of the present invention can collect the necessary data and the program can automatically calculate the maximum load for each sample without requiring estimates of the load from the plot.
3) Performing these tests, especially SCB test and IDEAL-CT tests, in a commonly available load frame is a challenge because the data acquisition rate is too slow for accurate results. Since the SCB test measures both the fracture and the brittleness, it requires high data acquisition rates of 20 Hz or greater. For brittle mixtures, the data acquisition rate may be as high as 100 Hz because the load rapidly decreases after the peak load because of the brittle response. In some embodiments, instruments of the present invention are capable of measuring data up to 200 Hz. In some other embodiments, instruments of the present invention are capable of measuring data at more than 200 Hz.
4) In the past, the displacement the sample undergoes while a force is applied is assumed to be constant since the plotter paper scrolls across with a constant rate. This may be or may not actually be the case depending on the stiffness of the material, the capabilities of the drive train, and compliance, i.e., flexibility, of the load frame. Therefore, it cannot be evaluated unless an independent displacement transducer is used to monitor the actual displacement. According to some embodiments, the present invention incorporates one or more, e.g., a set of, displacement transducers to accurately evaluate the loading rate. Furthermore, SCB and IDEAL-CT tests require measuring the actual (load-line) displacement.
5) Several tests require more information than the peak load. The area under the load versus displacement curve, which can be the area up to the peak load or the whole loading history, must be calculated for the SCB and IDEAL-CT tests. The area can be estimated using the trapezoidal rule using graph paper, which can introduce inaccuracies, or it can be calculated or determined using software. According to some embodiments, the present invention incorporates software to calculate the final results required for different tests. The software can operate on various devices such as a computer, or a portable electronic device employing, e.g., Android and IOS platforms. The final results can also be calculated in a controller or microprocessor on the fixture/jig and final results transferred to various devices such as a printer, plotter, computer, hand held device, tablet or a smart phone. This reduces the time required to calculate the result and eliminates any errors transcribing the data.

According to embodiments described herein, the present invention provides all the data acquisition and monitoring needed to perform several of the compression or tension performance tests required in the construction industry. The load or loading frame only has to provide the correct displacement rate and be able to supply the maximum loading forces required. The test apparatus includes a load cell that is positioned in such a way that if the test apparatus is not centered correctly under the loading frame there is little to no off center error introduced. The load cell can be positioned below or above the sample. Load cells are sensitive to the placement of the applied load. If the load is applied in a location that is different from the calibration procedure, the load could be read inaccurately. The load cell is configured within the test apparatus in such a way that it is protected from external forces or impulses other than the direct loading by constraining the movement by guide bars. This is explained in more detail below. Attached to the load cell are the amplification, microprocessor (or controller), and/or data acquisition electronic components that are a part of the test fixture. A computer, handheld device, embedded user interface, tablet, printer, plotter, and/or smartphone can connect directly or wirelessly to the electronic components to retrieve digitally stored data. Alternatively, computer, handheld device, embedded user interface, tablet, and/or smartphone can be connected or communicatively coupled to the electronic components to collect the load as a function of time or as a function of displacement and calculate, store and report the results. According to some embodiments, the present invention also includes the ability to measure the rate of deformation of the sample as force is applied. Displacement monitoring transducers may be mounted to each side of the cross bar of the test fixture or mounted to the base and referenced to the load frame so that deformation data and load cell data are sent to the electronic component(s) as the load is applied against the sample and it starts to compress. This data, force and displacement, may then be processed by the microprocessor (or controller) and results are sent to computer, handheld device, embedded user interface, tablet, printer, plotter, and/or smartphone. Force and displacement data can also be sent directly to a computer, handheld device, embedded user interface, tablet, and/or smartphone where it is immediately accessible to the user for viewing or for analyzing.

The application program that may be provided with the test fixture is capable of collecting, analyzing, calculating or determining, storing, and/or reviewing the data. The data collection and analysis can be performed by a handheld device, computer, tablet and/or smart phone operating in, for example, Android or IOS platform.

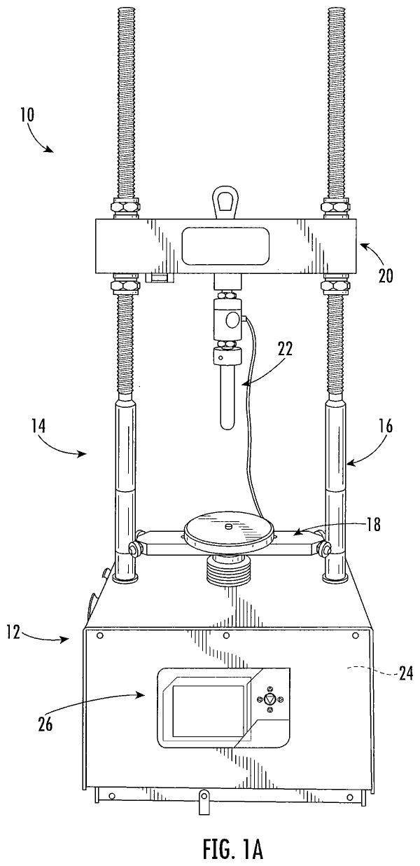

FIG. 1A illustrates an example load frame 10 that can be used with test fixtures or jigs of the present invention. The load frame 10 includes a housing 12. First and second guide bars 14, 16 extend upwardly from the housing 12. A platform 18 is between the first and second guide bars 14, 16. A cross bar 20 is also between the first and second guide bars 14, 16. A loading rod 22 extends downwardly from the cross bar 20. The loading rod 22 may be configured to move up and down away from and toward the platform 18 (e.g., under electric power, hydraulic power, pneumatic power, etc.). Additionally or alternatively, the platform 18 may be configured to move up and down away from and toward the loading rod 22 (e.g., under electric power, hydraulic power, pneumatic power, etc.). As described in more detail below, a test fixture or jig according to embodiments described herein can rest on the platform 18 and the loading rod 22 can move downwardly to apply a load at a prescribed rate to a test fixture or jig. In some embodiments, the test frame 10 includes a controller 24 and/or a display 26. The controller 24 may be configured to, for example, process load and displacement data as described herein and/or direct the display 26 to display the load and displacement data and other parameters as described herein.

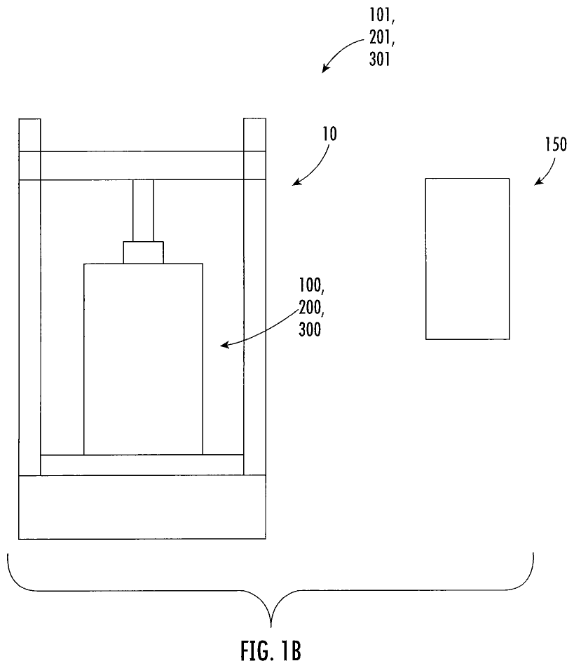

FIGS. 2A and 2B illustrate an embodiment of an instrument 100 (also referred to herein as a test fixture or jig) set up to perform an indirect tensile test using the Lottman breaking head configuration. The instrument 100 is capable of accurately measuring the load applied by a loading frame (e.g., the load frame shown in FIG. 1A) to a load plate or load platen 102 and impressed on sample 104. The instrument 100 includes a body 106 including a base 108, first and second guide bars 110, 112 extending upwardly from the base 108, an upper cross bar 114 extending between the first and second guide bars 110, 112, and a lower cross bar 116 extending between the first and second guide bars 110, 112. The instrument 100 can be placed into a loading frame (e.g., the load frame 10 shown in FIG. 1B) and centered properly in the loading frame by, for example, using a centering slot 118 in the base 108 of the instrument 100. This is done so that the center of the load plate 102 is in line with the center of the load frame loading ram or rod (see, e.g., the loading rod 22 in FIG. 1). When the load frame applies a load on the load plate 102, which is in contact with the upper cross bar 114, the load is transferred to the upper cross bar 114, which is guided by upper guide bearings 122 and 124 attached to upper cross bar 114 and that slide on guide bars 110 and 112, respectively. An upper press bar 126 is fastened to the underside of the upper cross bar 114 so that the load is further transferred to sample 104 through the upper press bar 126. The upper press bar 126 is designed and shaped to have the same curvature as the sample 104 at an upper contact surface 128 of the sample 104 and/or the upper press bar 126. The load is transferred through sample 104 and is constrained by a lower press bar 130, which is attached to the lower cross bar 116, and the upper press bar 126. The lower cross bar 116 is designed to be guided by lower guide bearings 132, 134 such that it is parallel to the base 108 during the application of a load and perpendicular to the load cell 136. The lower cross bar 116 transfers the load to the load cell 136, which may be embedded in the base 108 and may press against the lower cross bar 116.

During the test, the sample 104 is expected to break. In order to keep the upper cross bar 114 from falling once the sample breaks, stop collars 138 and 140 can be provided and may be adjustable (e.g., up and down along the guide bars 110 and 112, respectively).

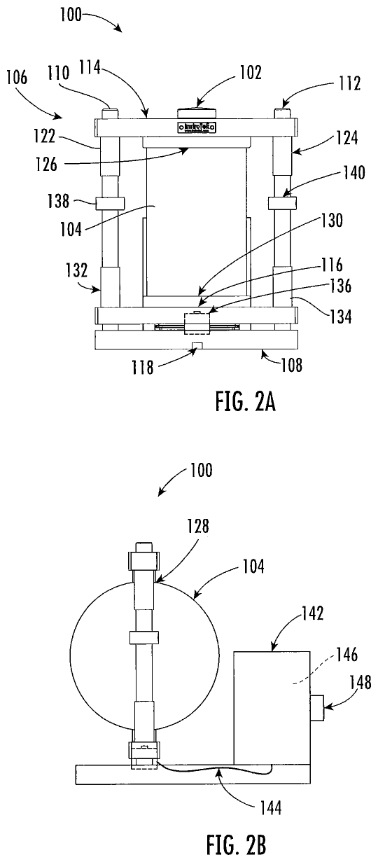

The load cell 136 produces an electrical signal proportional to the load and may be supplied power and monitored by load cell electronic components 142 through connector 144 which may be or include a cable. This signal may then be processed by a controller or microprocessor 146 (which may be one of the electronic components 142) and/or sent to a data acquisition system such as a computer, handheld device, tablet, printer, plotter, and/or smartphone through a transmitter or transceiver such as an electromagnetic computer connection 148. Those skilled in the art will observe that the communication to a data acquisition system may be through various medium, such as Bluetooth, infrared, or different types of cable configuration and protocols. FIG. 3 shows the instrument 100 configured as a Smart TSR connected to an electronic device such as a computer, tablet, or smartphone 150 for data acquisition purposes. In an embodiment, data is obtained from the Smart TSR or instrument 100 through the transmitter or transceiver such as an electromagnetic signal emitter 148 (FIG. 2B) through a signal 152 to a receiver or transceiver such as an electromagnetic receiver 154 of the electronic device 150 such as a computer, tablet, or smartphone. The electronic device 150 may include a display 158 for displaying data and/or results of the test. The electronic device 150 may include a controller 156 for processing data and/or controlling the display 158.

Still referring to FIG. 3, in some embodiments, a system 101 includes the instrument 100 and the electronic device 150. In some embodiments, the system 101 includes the instrument 100, the electronic device 150, and/or the load frame 10 (FIG. 1B).

FIGS. 4A and 4B show an embodiment of an instrument 200 (also referred to herein as a test fixture or jig) configured to perform an SCB test. The load is applied to the load plate or load platen 202 (e.g., using the load frame 10 shown in FIG. 1). The instrument 200 includes a body 206 including a base 208, first and second guide bars 210, 212 extending upwardly from the base 208, a load bar 214 extending between the first and second guide bars 210, 212, and a cross bar 216 extending between the first and second guide bars 210, 212. The load plate 202 is attached to the load bar 214 which rests on top of a load cell 218. The load cell 218 is positioned in or on the cross bar 216. The load bar 214 is constrained to remain parallel to the base 208 allowing only vertical motion aided by the guide bars 210, 212 and corresponding guide bushings 220, 222. Both the guide bars 210, 212 and the guide bushings 220, 222 are situated so that the horizontal plane of the load bar 214 is perpendicular to the button of the load cell 218. Under and attached to the cross bar 216 is a press bar 224 which makes contact with sample 204. The sample 204 rests on two round roll pins or rollers 226 situated at the opposing laterally spaced apart ends of the sample. Placement of the sample may be aided by a sample centering bar 228.

The roll pins 226 rest on one or more roll pin supports 230. As a load is applied to load plate 202, the sample 204 begins to deform in the center and the roll pins 226 have sufficient movement to allow the sample 204 to deform. Those skilled in the art will recognize that the allowable movement may additionally or alternatively be realized by utilizing grooves, springs, or a combination of grooves and springs.

As the load is applied to the load plate 202 (e.g., using the load frame 10 shown in FIG. 1) and the sample 204 begins to deform, the load plate 202, the load bar 214, the load cell 218, the cross bar 216, the guide bushings 220 and 222, and the press bar 224 move vertically down. This combination of elements 202, 214, 218, 216, 220 and 222, and 224 may be referred to herein as the cross bar assembly 240.

Displacement transducers 242 on each side of the cross bar 216 are used to measure the displacement of cross bar 216 as a function of time. By placing displacement transducers on each side of the cross bar 216 and averaging the two displacement transducers and combining the signals, any deviation from horizontal can be corrected. The displacement transducers 242 each have a displacement plunger 246 which rests on plunger shelf 248. The plunger shelves 248 are attached to the guide rods 210, 212 and are at rest relative to the motion of the cross bar 216 and cross bar assembly 240. As the cross 216 bar moves down, each displacement transducer plunger 246 is forced up and the displacement transducer 242 produces a signal proportional to the position of plunger 246 contained in displacement transducer 305 and is sampled at a periodic rate. Those skilled in the art will recognize that the same measurement may be accomplished with one displacement transducer attached to the jig similar to displacement transducers 242 either with or without corrections when one side of the cross bar moves different relative to the other. In addition, the load cell 218 also produces a signal proportional to the load and is also sampled at the same periodic rate. In an embodiment, the instrument 200 electronic components 250 (which may include a controller or microprocessor 256) have a circuit or mechanism to communicate with a data acquisition system through electromagnetic signals. Those skilled in the art will recognize that communication may also be established through a cable, infrared, Bluetooth, or other means.

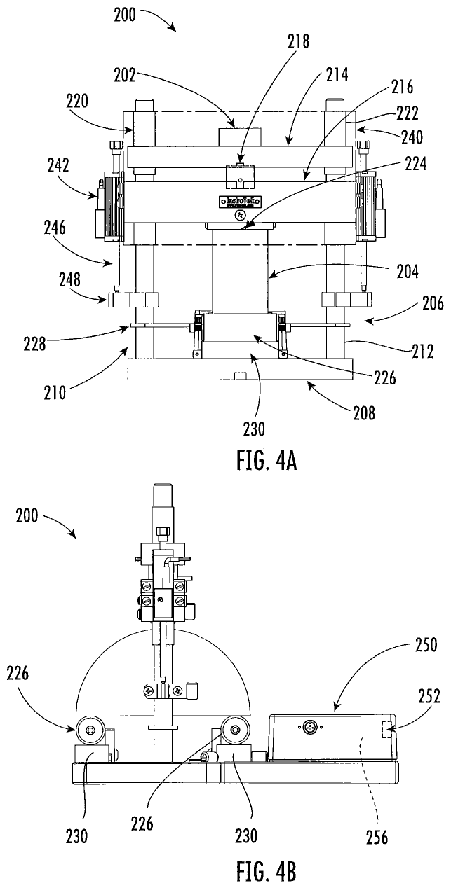

FIG. 5 shows the instrument 200 configured as a Smart SCB connected to the electronic device such as a computer, tablet, or smart phone 150 for data acquisition purposes. The Smart SCB electronic components may include a transmitter or transceiver such as an electromagnetic signal emitter 252 that transmits a signal 152, e.g., through an electromagnetic signal, that is acquired by electronic device 150 utilizing a receiver or transceiver such as an electromagnetic receiver 154 of the electronic device.

Still referring to FIG. 5, the system 201 may include the instrument 200 and the electronic device 150. In some embodiments, the system 201 includes the instrument 200, the electronic device 150, and/or the load frame 10 (FIG. 1B).

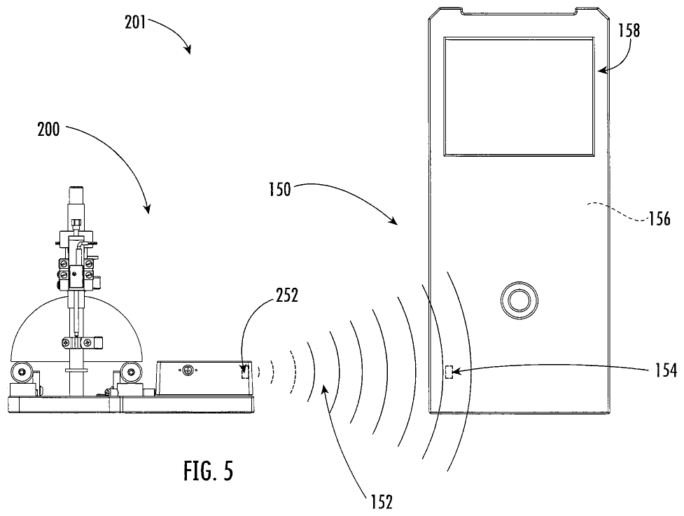

FIGS. 6A and 6B show an instrument 300 (also referred to herein as a test fixture or jig) configured to perform a Marshall Stability test according to some embodiments. The instrument 300 is the same or substantially the same as the instrument 100 discussed with respect to FIGS. 2A, 2B, and 3, except for upper press bar 326 and lower press bar 330, which may surround the circumference or a major portion of the circumference of a cylindrical asphalt sample 304. The instrument 300 is placed into a loading frame centered with the load plate or load platen 302 centered under the load frame ram (for example, the loading rod 22 in FIG. 1). The instrument 300 may include a body 306 including a base 308, first and second guide bars 310, 312 extending upwardly from the base 308, an upper cross bar 314 extending between the first and second guide bars 310, 312, and a lower cross bar 316 extending between the first and second guide bars 310, 312. Centering of instrument 300 may be aided by centering slot 318 in base 308. A load is applied to load plate 302 and is transferred to sample 304 through the upper cross bar 314 and upper press bar 326 which makes contact with the sample (outer) surface 328. The upper cross bar 314 moves vertically downward aided by the guide bars 310, 312 and upper guide bearings 322, 324. The sample 304 is further constrained by the lower press bar 330 which is in contact with sample (outer) surface 328 and attached to the lower cross bar 316. The load is transferred from lower press bar 330 to lower cross bar 316 which rests on load cell 336. The lower cross bar 316 is constrained to be parallel to the base 308 and perpendicular to the button of the load cell 336 aided by lower guide bearings 332 and 334 which ride on guide bars 310 and 312, respectively. Load cell 336 is connected to load cell electronic components 342 by load cell electronic cable 344. The electronic components 342 (which may include controller or microprocessor 346) may then transmit data to a data acquisition system through data acquisition connection 348, such as a transmitter or transceiver 348 that may transmit the data to the electronic device 150 (FIG. 3). Like with the instruments 100 and 200, the data may be processed at the instrument 300 (e.g., using the controller 346) or at the electronic device 150.

Like with the instruments 100 and 200, a system may include the instrument 300 and the electronic device 150. In some embodiments, the system 301 includes the instrument 300, the electronic device 150, and/or the load frame 10 (FIG. 1B).

FIG. 7 shows an example of the data 400 acquired from the instrument 100, 200, or 300 and the analysis that may be carried out. The data may be acquired at a rate of between 20 and 200 Hz and, in some embodiments, at least 100 Hz, and plotted on a graph as load 403 versus displacement 404. The peak or maximum load 401 is important for determining the strength of the sample. The area under the curve 402 represents the fracture energy required to break the specimen. The slope after the peak load 405 is an indication of the brittleness of the sample. Parameters 401, 402, and 405 are used to calculate performance values for the respective tests.

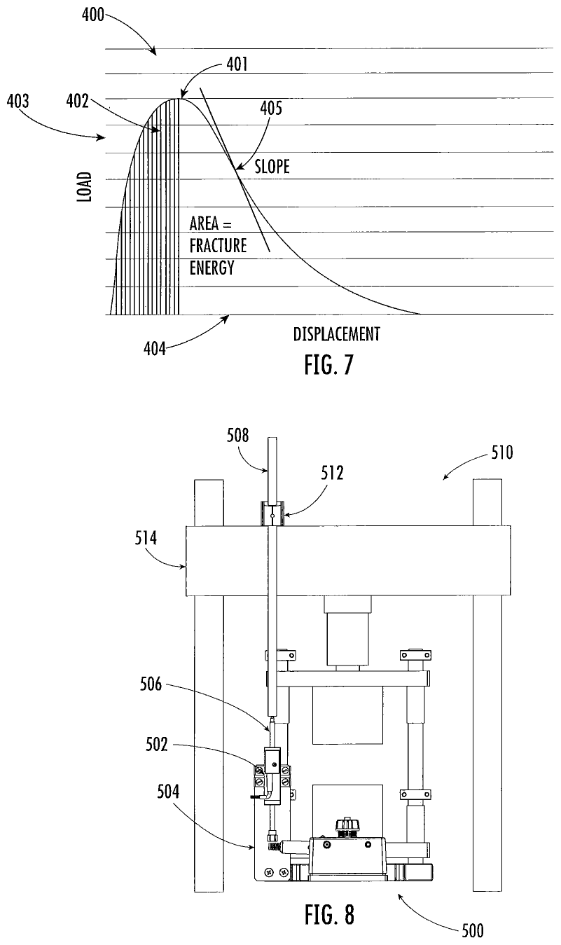

FIG. 8 shows another embodiment of an instrument 500 (also referred to herein as a test fixture or jig) situated in loading frame 510 and fitted with a single LVDT 502 and capable of performing the tests mentioned previously in other embodiments. The LVDT 502 is supported by fixture or support 504 which is part of or attached to the body of the jig 500. The LVDT plunger 506 is adjacent and/or rests on the lower end of rod 508. Rod 508 is fixed to a clamp 512 which rests on cross bar 514 of the loading frame 510. As the jig is moved up the LVDT plunger 506 is depressed by stationary rod 508 indicating the amount of movement.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention. Therefore, it is to be understood that the foregoing is illustrative of the present invention and is not to be construed as limited to the specific embodiments disclosed, and that modifications to the disclosed embodiments, as well as other embodiments, are intended to be included within the scope of the invention.

1. A system for evaluating properties of an asphalt sample, the system comprising:
a load frame comprising a platform and a loading rod, wherein one of the platform and the loading rod is configured to translate up and down away from and toward the other one of the platform and the loading rod; and
a test fixture comprising:
a base configured to rest on the platform of the load frame;
first and second spaced apart vertical guide bars extending upwardly from the base;
a horizontal cross bar above the base and extending between the first and second guide bars, wherein the asphalt sample is configured to be held between the base and the cross bar;
a load plate above the cross bar, the load plate configured to receive the loading rod of the load frame to apply a load to the asphalt sample;
a load cell above the base and configured to measure the applied load and to generate corresponding load electrical signals; and
a controller configured to receive the load electrical signals.

2. The system of claim 1 wherein the cross bar is an upper cross bar, the test fixture further comprising a horizontal lower cross bar above the base and extending between the first and second guide bars, wherein:
the load plate is on the upper cross bar; and
the load cell is between the base and the lower cross bar.

3. The system of claim 2, the test fixture further comprising a lower press bar at an upper portion or surface of the lower cross bar and an upper press bar at a lower portion or surface of the upper cross bar, wherein the asphalt sample is configured to be received between the lower press bar and the upper press bar.

4. The system of claim 3, wherein:
the asphalt sample is cylindrical; and
the lower press bar and the upper press bar are each arcuate to surround at least a major portion of a circumference of the asphalt sample.

5. The system of claim 3, the test fixture further comprising first and second upper guide bearings each coupled to the upper cross bar, the first upper guide bearing surrounding the first guide bar and the second upper guide bearing surrounding the second guide bar, the first and second upper guide bearings configured to allow vertical movement of the upper guide bar upon application of the load.

6. The system of claim 3, the test fixture further comprising a transmitter or transceiver, wherein the controller is configured to, using the transmitter or transceiver, wirelessly transmit load data associated with the load electrical signals to an electronic device.

7. The system of claim 6 further comprising the electronic device, wherein the electronic device is configured to display the load data versus displacement data and optionally a peak load to break the asphalt sample.

8. The system of claim 1, the test fixture further comprising a horizontal load bar above the cross bar and extending between the first and second guide bars, wherein:
the load plate is on the load bar; and
the load cell is between the cross bar and the load bar.

9. The system of claim 8 wherein:
the asphalt sample is semi-cylindrical with a circumference comprising a curved portion and a flat portion;
the test fixture further comprises:
a press bar at a lower portion or surface of the cross bar and configured to engage the curved portion of the circumference of the asphalt sample; and
first and second rollable pins above the base and configured to engage the flat portion of the circumference of the asphalt sample thereon.

10. The system of claim 8, the test fixture further comprising a first displacement transducer coupled to a first side of the cross bar adjacent the first guide bar and a second displacement transducer coupled to a second side of the cross bar adjacent the second guide bar, the first and second displacement transducers configured to measure a displacement of the cross bar as the load is applied by the load frame and to generate corresponding displacement electrical signals.

11. The system of claim 10, wherein the load cell is configured to generate the load electrical signals and the first and second displacement transducers are configured to generate the displacement electrical signals at a rate of 40 Hz or greater.

12. The system of claim 10 wherein:
the first displacement transducer comprises a first plunger and the second displacement transducer comprises a second plunger;
a first shelf is coupled to the first guide bar and a second shelf is coupled to the second guide bar; and
the first plunger rests on the first shelf and the second plunger rests on the second shelf.

13. The system of claim 10, the test fixture further comprising a transmitter or transceiver, wherein:
the controller is configured to receive the displacement electrical signals from the first and second displacement transducers; and
the controller is configured to, using the transmitter or transceiver, wirelessly transmit displacement data associated with the displacement electrical signals and load data associated with the load electrical signals to an electronic device.

14. The system of claim 13 further comprising the electronic device comprising a controller and a display, wherein the controller of the test fixture or the controller of the electronic device is configured to determine a fracture energy of the asphalt sample based on the load data and the displacement data, to determine a brittleness of the asphalt sample based on the load data and the displacement data, and the controller of the electronic device is configured to direct the display to display the load data, the displacement data, the fracture energy of the asphalt sample, and/or the brittleness of the asphalt sample.

15. A test fixture for use with a load frame and for evaluating properties of an asphalt sample, the test fixture comprising:
a base configured to rest on a platform of the load frame;
first and second spaced apart vertical guide bars extending upwardly from the base;
a horizontal cross bar above the base and extending between the first and second guide bars, wherein the asphalt sample is configured to be held between the base and the cross bar;
a load plate above the cross bar, the load plate configured to receive a loading rod of the load frame to apply a load to the asphalt sample;
a load cell above the base and configured to measure the load and to generate corresponding load electrical signals; and
a controller configured to receive the load electrical signals.

16. The test fixture of claim 15 wherein the cross bar is an upper cross bar and the load plate is on the upper cross bar, the test fixture further comprising:
a horizontal lower cross bar above the base and extending between the first and second guide bars, wherein the load cell is between the base and the lower cross bar; and
a lower press bar at an upper portion or surface of the lower cross bar and an upper press bar at a lower portion or surface of the upper cross bar, wherein the asphalt sample is configured to be received between the lower press bar and the upper press bar; and
a transmitter or transceiver, wherein the controller is configured to, using the transmitter or transceiver, wirelessly transmit load data associated with the load electrical signals to an electronic device such that the electronic device can store and/or display the load data versus displacement data and optionally a peak load to break the asphalt sample.

17. The test fixture of claim 15 further comprising:
a horizontal load bar above the cross bar and extending between the first and second guide bars, wherein the load plate is on the load bar and the load cell is between the cross bar and the load bar;
at least one displacement transducer coupled to the cross bar and configured to measure a displacement of the cross bar as the load is applied by the load frame and to generate corresponding displacement electrical signals; and
a transmitter or transceiver;
wherein:
the controller is configured to receive the displacement electrical signals from the first and second displacement transducers;
the controller is configured to, using the transmitter or transceiver, wirelessly transmit displacement data associated with the displacement electrical signals and load data associated with the load electrical signals to an electronic device such that the electronic device can store and/or display the load data, the displacement data, a fracture energy of the asphalt sample based on the load data and the displacement data, and/or a brittleness of the asphalt sample based on the load data and the displacement data; and
the controller is configured to, using the transmitter or transceiver, wirelessly transmit the displacement data and load data to the electronic device at a rate of at least 40 Hz.

18. A method of evaluating mechanical properties of an asphalt test sample, the method comprising:
providing a test fixture comprising a base, first and second spaced apart vertical guide bars extending upwardly from the base, a horizontal cross bar above the base and extending between the first and second guide bars, a load plate above the cross bar, a load cell above the base, a controller in communication with the load cell, and optionally a transmitter in communication with the controller;
positioning an asphalt sample between the base and the cross bar of the test fixture;
positioning the test fixture in a load frame by resting the base of the test fixture on a platform of the load frame;
loading the asphalt sample by receiving a loading rod of the test frame on the load plate of the test fixture;
generating load electrical signals using the load cell in response to the loading step;
receiving the load electrical signals at the controller; and
transmitting, optionally wirelessly, load data associated with the load electrical signals to an electronic device using the controller and optionally the transmitter.

19. The method of claim 18 further comprising displaying and/or storing at the electronic device the load data versus displacement data and optionally a peak load to break the asphalt sample.

20. The method of claim 18 wherein the test fixture further comprises a first displacement transducer coupled to a first side of the cross bar adjacent the first guide bar and a second displacement transducer coupled to a second side of the cross bar adjacent the second guide bar, the method further comprising:
generating displacement electrical signals using the first and second displacement transducers in response to the loading step;
receiving the displacement electrical signals at the controller;
transmitting, optionally wirelessly, displacement data associated with the displacement electrical signals to the electronic device using the controller and optionally the transmitter; and
displaying and/or storing at the electronic device the load data, the displacement data, a fracture energy of the asphalt sample based on the load data and the displacement data, and/or a brittleness of the asphalt sample based on the load data and the displacement data,
wherein the steps of transmitting the load data and transmitting the displacement data are carried out at a rate of 40 Hz or more.
